(19) United States
(12) Patent Application Publication
(10) Pub. No.: US 2014/0341988 A1
(43) Pub. Date: Nov. 20, 2014

(54) ORALLY DISPERSIBLE DRUG FORMULATIONS

(71) Applicant: APR APPLIED PHARMA RESEARCH SA, Balerna (CH)

(72) Inventors: Alberto REINER, Como (IT); Giorgio REINER, Como (IT)

(73) Assignee: APR APPLIED PHARMA RESEARCH SA, Balerna (CH)

(21) Appl. No.: 14/277,941

(22) Filed: May 15, 2014

Related U.S. Application Data
(60) Provisional application No. 61/823,620, filed on May 15, 2013.

(51) Int. Cl. A61K 31/616; A61K 31/138; A61K 31/485; A61K 31/192; A61K 9/20; A61K 31/167; A61K 31/137; A61K 31/522; A61K 45/06; A61K 31/09; A61K 31/4545

(52) U.S. Cl. CPC A61K 31/616; A61K 31/09; A61K 31/138; A61K 31/485; A61K 31/192; A61K 31/4545; A61K 31/167; A61K 31/137; A61K 31/522; A61K 45/06; A61K 9/2095; A61K 9/2059; A61K 9/2018
USPC 424/465; 514/718; 514/648; 514/289; 514/570; 514/290; 514/630; 514/653; 514/161; 514/263.34; 514/165

(57) ABSTRACT

Pharmaceutical dosage forms, particularly dosage forms in granular form having good palatability and capable of rapidly and completely dispersed in the mouth when orally administered.

### CROSS-REFERENCE TO RELATED APPLICATIONS AND CLAIM TO PRIORITY

This application claims priority to U.S. Provisional Application Ser. No. 61/823,620 filed May 15, 2013. This application is incorporated herein by reference.

### FIELD OF THE INVENTION

The present invention relates to pharmaceutical dosage forms, particularly to dosage forms in granular form having good palatability and capable of rapidly and, when necessary, completely dissolving or dispersing in the mouth when orally administered.

### BACKGROUND OF THE INVENTION

The delivery of drugs via oral powders is well known and includes, for example, headache powders sold under the brand names BC® Powder and Goody's® Headache Powder. However, the dosage forms have not gained widespread acceptance due principally to the poor taste of the product, and length of time the medication is exposed to the taste buds and olfactory senses. This is especially true for children's medicines, given the sensitivity of most children to poor tasting medications.

It is therefore an object of the present invention to provide a granular dosage form in which the active ingredient dissolves or disperses rapidly when delivered to the oral cavity, with or without ingestion of any additional liquids.

It is a further object of the present invention to provide a granular dosage form that is palatable to the senses when dissolved or dispersed in the mouth, preferably by coating the active ingredient.

A still further object of the invention is to provide manufacturing processes for such dosage forms.

### SUMMARY OF THE INVENTION

It has unexpectedly been discovered that by integrating a polymeric agent normally used to prepare delayed release tablets or to coat tablets into a granular composition, along with a rapidly disintegrating diluent and optional flavoring and sweetening agents, a granular composition can be obtained that simultaneously dissolves or disperses rapidly in the mouth and overcomes the poor taste associated with many active drug ingredients without affecting in a significant way the dissolution profile. A particular preferable polymeric agent is one that acts as a coating agent used in delayed release formulations. One example of a suitable coating agent is an enteric coating agent that resists dissolution in the acidic environment of the stomach, and only dissolves in the higher pH environment of the small intestine.

In one embodiment, therefore, the invention provides a method of making an orally dispersible formulation comprising:
i) adding at least one active ingredient to a high speed mixer and shearing said active ingredient at a first rate;
ii) adding a granulating solution comprising a solvent and a coating agent to said high speed mixer while shearing said active ingredient to form a wet mixture;
iii) shearing said wet mixture in said high speed mixer at a second rate greater than said first rate;
iv) adding a diluent plus disintegrating agent and optionally other excipients (e.g. buffering agents, flavors, flavor enhancers) to said wet mixture while shearing said wet mixture at said second rate until a first granular mixture forms; and optionally passing the granular mixture through a suitable sieve; and
v) drying said first granular mixture.

In another embodiment the invention provides a method of making an orally dispersible formulation comprising:
i) adding a diluent and a disintegrating agent to a high speed mixer and shearing said diluent and disintegrating agent at a first rate to form a preliminary mixture;
ii) adding an active ingredient, and a coating agent and optionally other excipients (e.g. buffering agents, flavors, flavor enhancers), to said preliminary mixture while shearing said preliminary mixture, active ingredient and coating agent at said first rate to form a wet mixture, wherein the coating agent is dissolved in a solvent to form a granulating solution and wherein the active ingredient is dissolved or in suspension in the granulating solution;
iii) shearing said wet mixture at a second rate greater than said first rate until a first granular mixture forms and optionally passing the granular mixture through a suitable sieve; and
iv) drying said granular mixture.

The final formulation can be composed of the first granular mixture optionally along with extragranular agents such as sweeteners and flavorings to improve the quality of the finished product. In addition, the granular mixture can be compressed into tablets and administered as an orally disintegrating tablet. Additionally, the solvent used in the granulating solution can be ethanol, water, acetone, or another suitable solvent or combination thereof.

In still another embodiment the invention provides an orally dispersible granular mixture comprising a plurality of granules wherein each of said granules comprises an active ingredient, a diluent, a disintegrating agent, a coating agent, and optionally other excipients (e.g. buffering agents, flavors, flavor enhancers), each uniformly distributed throughout said granules, wherein said granular mixture optionally substantially completely dissolves or disperses within seconds to minutes in the mouth when orally administered. In yet another embodiment the granular mixture completely dissolves or disperses in the mouth, but the active ingredient does not dissolve completely into solution.

Finally, the invention provides a method of delivering an active ingredient to a human patient comprising administering to said patient the granular mixture described above.

Additional advantages of the invention are set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWING

FIG. 1 depicts the dissolution profile of ibuprofen from prototypes bkT027/133 (Eudragit), bkT027/15 (HPMCP), and bkT032/63 (HPMCP) in comparison with the dissolution profile of the Nurofen 200 mg as a reference market product.

FIG. 2 depicts the dissolution profile of loratadine ODP granulate batches bkT027/19 (contained in finished product batch bkT027/51) and bkT027/21 (contained in finished product batch bkT027/55), which are the prototypes with (bkT027/21) and without (bkT027/19) HPMCP (both without the extra granules). The dissolution media had a pH of 1.2.

FIG. 3 depicts the dissolution profile of the ketoprofene lysine salt prototype bkT032/55. The dissolution media had a pH of 4.5.

FIG. 4 depicts the dissolution profile of acetaminophen, guaifenesin, dextromethorphan and phenylephrine from the acetaminophen/dextromethorphan/guaifenesin/phenylephrine prototype bkT027/124. The dissolution media had a pH of 1.2.

FIG. 5 depicts the dissolution profile of acetaminophen, caffeine and ASA from the acetaminophen/ASA/Caffeine prototype bkT027/128. The dissolution media had a pH of 1.2.

FIGS. 6A-D depict the dissolution profile of acetaminophen from two acetaminophen and diphenhydramine HCl prototypes: bkT027/108 and bkT027/129. FIG. 6A depicts the dissolution profile of the acetaminophen and diphenhydramine HCl from bkT027/108 in a dissolution media of pH 1.2. FIG. 6B depicts the dissolution profile of the acetaminophen and diphenhydramine HCl from bkT027/108 in a dissolution media of pH 4.5. FIG. 6C depicts the dissolution profile of the acetaminophen and diphenhydramine HCl from bkT027/108 in a dissolution media of pH 6.8. FIG. 6D depicts the dissolution profile of the acetaminophen and diphenhydramine HCl from bkT027/129 in a dissolution media of pH 1.2.

### DETAILED DESCRIPTION OF THE INVENTION

### Definition and Use of Terms

When the singular forms “a,” “an” and “the” or like terms are used herein, they will be understood to include plural referents unless the context clearly dictates otherwise. Thus, for example, reference to “a hydrocarbon” includes mixtures of two or more such hydrocarbons, and the like. The word “or” or like terms as used herein means any one member of a particular list and also includes any combination of members of that list.

When used herein the term “about” or “ca.” will compensate for variability allowed for in the pharmaceutical industry and inherent in pharmaceutical products, such as differences in product strength and bioavailability due to manufacturing variations and time-induced product degradation. The term allows for any variation which in the practice of pharmaceuticals would allow the product being evaluated to be considered pharmaceutically equivalent or bioequivalent, or both if the context requires, to the recited strength of a claimed product. It will be understood that all numeric values expressed in this document can be prefaced by the term “about.”

Throughout the description and claims of this specification, the word “comprise” and variations of the word, such as “comprising” and “comprises,” means “including but not limited to,” and is not intended to exclude, for example, other additives, components, integers or steps.

When a range of values can be used to describe a particular regiment, it will be understood that the range can be defined by selectively combining any one of the lower end of variables described in the specification with any one of the upper end of variables described in the specification that is mathematically possible.

The terms “treating” and “treatment,” when used herein, refer to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, “therapeutically effective amount” refers to an amount sufficient to elicit the desired biological response. The therapeutically effective amount or dose will depend on the age, sex and weight of the patient, and the current medical condition of the patient. The skilled artisan will be able to determine appropriate dosages depending on these and other factors in addition to the present disclosure.

As used herein, “disperse” or “dispersing” means that the dosage form is distributed in the mouth, regardless of whether a solution is formed.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use. “Pharmaceutically acceptable salts” means salts that are pharmaceutically acceptable, as defined above, and which possess the desired pharmacological activity.

When a weight of an active ingredient is given without reference to the free base or salt of the active ingredient, it will be understood that the weight can refer to the weight of the free base or the weight of the entire salt.

### Discussion

As described above, the invention provides orally dispersible formulations that overcome the poor taste of many active ingredients, methods of making such formulations, and methods of administering said formulations.

Practically any active ingredient can be used in the methods and formulations of the present invention, although those exhibiting poor palatability are especially preferred. In a preferred embodiment, the active ingredient is selected from the group consisting of guaifenesin, diphenhydramine, dextromethorphan, ibuprofen, ketoprofen, loratadine, phenylephrine, caffeine and acetylsalicylic acid, and combinations thereof, wherein any of said active ingredients can be present as a pharmaceutically acceptable salt. In another preferred embodiment, the active ingredient is selected from the group consisting of non-steroidal medications, anti-inflammatory medications, allergy medications, analgesics, antacids, anticholinergics, antidiarrheals, antiemetics, antiflatulents, antihistamines, antirheumatics, antitussives, bronchodilators, decongestants, expectorants, laxatives, sleep aids, sedatives, smoking deterrents, stimulants and stomach acidifiers, and combinations thereof, wherein any of the active ingredients can be present as its pharmaceutically acceptable salt.

Numerous coating agents can be used in the invention, but the coating agent is preferably one which acts as a coating agent in conventional delayed release oral formulations, including polymers for enteric coating.

Preferred examples include hypromellose phthalate (hydroxy propyl methyl cellulose phthalate; HPMCP); hydroxypropylcellulose (HPC; such as KLUCEL®); ethylcellulose (such as ETHOCEL®); and methacrylic acid and methyl methacrylate (MAA/MMA; such as EUDRAGIT®), as described in the following paragraphs.

HPMCP is the monophthalic acid ester of hypromellose (hydroxypropyl methylcellulose) and is widely used as an enteric coating agent by the pharmaceutical industry. For the prototypes described herein, HPMCP-55 was used.

KLUCEL® (hydroxypropylcellulose; HPC) is a nonionic water-soluble cellulose ether with a versatile combination of properties. HPC has a variety of applications: low molecular weight grades provide unmatched, efficient tablet binding and adhesive and elastic tablet coating. The breadth of viscosity grades can be used for wide ranging applications for modified release including matrix tablets. For the prototypes described herein the EXF Pharm fine grade was used.

ETHOCEL® (Ethylcellulose) is derived from cellulose so its backbone is based on repeating anhydroglucose units. The number of units and the degree of ethoxy substitution determine the ethylcellulose type: for the prototypes described herein the Ethocel® Standard 10 Premium was used. These polymers can provide a versatile diffusion barrier whose properties can be modified by film thickness, modifying the solvent(s) used and the molecular weight of ethylcellulose. They can control the release of an active ingredient.

EUDRAGIT® S 100 is an anionic copolymer based on methacrylic acid and methyl methacrylate (MAA/MMA). This polymer dissolves at pH 7.0 and is commonly used for the granulation of drug substances in powder form for controlled release.

EUDRAGIT®L30 D55 is the acqueous dispersion of anionic polymers with methacrylic acid as functional group. The compound dissolves at pH 5.5 and is commonly used for the granulation of drug substances in powder form for controlled release and for effective and stable enteric coatings with a fast dissolution in the upper Bowel.

The formulation also preferably includes at least one disintegrating agent, preferably a super disintegrant, as well as diluent. One example of a diluent is a bulking agent such as a polyalcohol. A preferred combination of bulking agent and disintegrant is marketed as PEARLITOL FLASH®, is a ready to use mixture of mannitol and maize starch (mannitol/maize starch). In general, any polyalcohol bulking agent can be used when coupled with a disintegrant or a super disintegrant agent such as maize starch. Additional disintegrating agents include, but are not limited to, agar, calcium carbonate, maize, potato or tapioca starch, alginic acid, alginates, certain silicates, and sodium carbonate. A list of suitable super disintegrating agents include, but are not limited to crospovidone, croscarmellose sodium, AMBERLITE (Rohm and Haas, Philadelphia, Pa.), and sodium starch glycolate.

Other components/excipients that can be added to the granules include. For example, sweeteners, flavors, buffering agents, and flavor enhancers to make the dosage form more palatable. Sweeteners include, but are not limited to, fructose, sucrose, glucose, maltose, mannose, galactose, lactose, sucralose, saccharin, aspartame, acesulfame K, and neotame. Common flavoring agents and flavor enhancers that may be included in the formulation of the present invention include, but are not limited to, maltol, vanillin, ethyl vanillin, menthol, citric acid, fumaric acid, ethyl maltol and tartaric acid.

Buffering agents may be added to maintain a desired pH value or pH range in which the granules are dispersed. Suitable buffering agents are known to those skilled in the art and should be selected based on its ability to disperse the granules within the mouth. Either inorganic buffering agents (e.g., phosphate, carbonate, etc.) or organic buffering agents (e.g., citrate, acetate, Tris, MOPS, MES, HEPES, etc.) may be used.

Different types of granules can also be mixed with the active-containing granulates to improve the properties of the formulation even further, referred to herein as “extragranulates.” Extragranulates include, for example, uncoated active ingredient/s, sweeteners, diluents, flavors, buffering agents and flavor enhancers. These can be mixed in as powders or as granulates themselves, produced according to common granulating processes.

Granular mixtures containing the same active ingredient but different coating agents, disintegrating agents, and diluents can be combined to form the final dosage form. Such dosage forms may be useful to control the release of the active ingredient.

Granular mixtures each containing different active ingredients (as well as different overall ingredients) can be combined to create the final dosage form.

The granular formulation dissolves or disperses in the mouth within about 5, 10, 15, 20, 25, 30, 35, 40, 45, or 50 seconds or about 1, 2, 3, 4, or 5 minutes. In another embodiment the granular formulation dissolves or disperses in the mouth between 1 to 10 seconds, 1 to 30 seconds, 1 to 45 seconds, 1 to 60 seconds, 1 to 2 minutes, or 1 to 5 minutes.

These powders can also be compressed into tablets or filled into capsules.

The granular formulation disperses rapidly at the pH of saliva in the mouth, which generally is from 6.2 to 7.4. In other embodiments, the granular formulation dissolves or disperses rapidly over a range of pH values, for example, from 1 to 10; 3 to 10; 4 to 9; 5 to 8; 1 to 4; 3 to 6; 7 to 10; 7 to 9; or 7 to 8.

The present invention provides oral dosage forms that are formulated or administered for gastrointestinal absorption of the active pharmaceutical agent, and that are bioequivalent to and interchangeable with existing orally administered drug products. These dosage forms are quickly disintegrated in the mouth when exposed to saliva; and they are absorbed predominantly through the gastrointestinal tract. Most importantly, these dosage forms are specially formulated to be bioequivalent to or have greater bioavailability than existing orally administered dosage forms that contain the same amount of active pharmaceutical agent or a pharmaceutically acceptable salt thereof in said defined amount (i.e. a “reference product”).

Bioequivalence testing typically requires an in vivo test in humans in which the concentration of the active ingredient or active moiety, and, when appropriate, its active metabolite(s), in whole blood, plasma, serum, or other appropriate biological fluid is measured as a function of time. Defined as relative bioavailability (“BA”), bioequivalence (“BE”) involves a comparison between a test and reference drug product. Although BA and BE are closely related, BE comparisons normally rely on (1) a criterion, (2) a confidence interval for the criterion, and (3) a predetermined BE limit.

A standard in vivo BE study design is based on the administration of either single or multiple doses of the test and reference products to healthy subjects on separate occasions, with random assignment to the two possible sequences of drug product administration. Statistical analysis for pharmacokinetic measures, such as area under the curve (AUC) and peak concentration (Cmax), is preferably based on the so-called “two one-sided tests procedure” to determine whether the average values for the pharmacokinetic measures determined after administration of the test and reference products are comparable. This approach is termed average bioequivalence and involves the calculation of a 90% confidence interval for the ratio of the averages (population geometric means) of the measures for the test and reference products. To establish BE, the calculated confidence interval should fall within a BE limit, i.e. 80-125% for the ratio of the product averages. Further detail regarding BE procedures can be found in FDA's July 1992 Guidance Document entitled “Statistical Procedures for Bioequivalence Studies Using a Standard Two-Treatment Crossover Design,” the contents of which are incorporated herein by reference.

### EXAMPLES

The following examples are put forth so as to provide those of ordinary skill in the art with a complete disclosure and description of how the methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.) but some errors and deviations should be accounted for.

### Example 1

### Selection of Coating Agents and Preliminary Evaluation of the Coating Effects of Active Ingredient Release

In order to demonstrate the advantages of the taste masking technology of the present invention, three different drugs characterized by a bad taste (Guaifenesin, Dextromethorphan HBr, and Diphenhydramine HCl) were evaluated for taste and dissolution properties when combined with mannitol/maize starch and granulation using different coating agents.

The mixtures tested were prepared as follows:
Mechanical mixing of the drug with mannitol/maize starch in a mixing machine;
Granulation of the drug/mannitol/maize starch combination using a solution of EtOH/water/other type of solvent (depending by the polymer used);
The EtOH/water/other type of solvent solution further contained a coating agent selected from HPMCP, KLUCEL® HPC, ETHOCEL® ethylcellulose and MAA/MMA EUDRAGIT® (two different types).

Dissolution profiles were determined for all the prototypes using different media (pH 1.2, 4.5, 6.8) prepared according to USP methods and tested in order to verify the effect of each coating agent at each pH on the drug release. In order to demonstrate the advantages of the different polymers, the taste of all the prototypes was tested.

### Example 1a

### Guaifenesin

### Combinations

| | A | B | C | D | E | F |
|---|---|---|---|---|---|---|
| Guaifenesin | 20.0 g | 20.0 g | 20.0 g | 20.0 g | 20.0 g | 20.0 g |
| PEARLITOL | 20.0 g | 20.0 g | 20.0 g | 20.0 g | 20.0 g | 20.0 g |
| FLASH ® | | | | | | |
| EtOH (90%) | — | 6.0 g | — | — | — | — |
| Solution at | — | — | 8.5 g | — | — | — |
| 10% of | | | | | | |
| HPMCP in | | | | | | |
| EtOH (90%)* | | | | | | |
| Solution at | — | — | — | 8.5 g | — | — |
| 10% of | | | | | | |
| KLUCEL ® in | | | | | | |
| EtOH (90%)* | | | | | | |
| Solution at | — | — | — | — | 8.5 g | — |
| 10% of | | | | | | |
| ETHOCEL ® | | | | | | |
| in EtOH | | | | | | |
| (90%)* | | | | | | |
| Solution at | — | — | — | — | — | 8.5 g |
| 10% of | | | | | | |
| EUDRAGIT ® | | | | | | |
| in EtOH | | | | | | |
| (90%)* | | | | | | |

*EtOH is eliminated during granulate desiccation

### Dissolution Tests

Each of the foregoing guaifenesin formulations was tested according to the protocol described below, and the time to reach a release higher than 85% was determined.
Analytical Equipment: UV
Apparatus: Paddle (50 rpm)
Mediums: pH 1.2/pH 4.5/pH 6.8 (prepared according to the USP)
Wavelength: 276 nm
Volume: 400 ml
Withdrawal times: 2.5, 5, 10, and 15 minutes

| | Time (min) for a release > 85% (Average of 4 units) | | |
|---|---|---|---|
| Combinations | Medium pH 1.2 | Medium pH 4.5 | Medium pH 6.8 |
| A | 2.5 | 2.5 | 2.5 |
| B | 2.5-5 | 2.5 | 5-10 |
| C | 2.5 | 5 | 2.5 |
| D | 10 | 2.5 | 10-15 |
| E | 10 | 5 | 2.5 |
| F | 2.5 | 5-10 | 5 |

### Example 1b

### Diphenhydramine HCl

### Combinations

| | A | B | C | D | E | F |
|---|---|---|---|---|---|---|
| Diphen- | 5.0 g | 5.0 g | 5.0 g | 5.0 g | 5.0 g | 5.0 g |
| hydramine | | | | | | |
| HCl | | | | | | |
| PEARLITOL | 50.0 g | 50.0 g | 50.0 g | 50.0 g | 50.0 g | 50.0 g |
| FLASH ® | | | | | | |
| EtOH (90%) | — | 10.0 g | — | — | — | — |
| Solution at | — | — | 11.5 g | — | — | — |
| 10% of | | | | | | |
| HPMCP in | | | | | | |
| EtOH (90%)* | | | | | | |
| Solution at | — | — | — | 11.5 g | — | — |
| 10% of | | | | | | |
| KLUCEL ® in | | | | | | |
| EtOH (90%)* | | | | | | |
| Solution at | — | — | — | — | 11.5 g | — |
| 10% of | | | | | | |
| ETHOCEL ® | | | | | | |
| in EtOH | | | | | | |
| (90%)* | | | | | | |
| Solution at | — | — | — | — | — | 11.5 g |
| 10% of | | | | | | |
| EUDRAGIT ® | | | | | | |
| in EtOH | | | | | | |
| (90%)* | | | | | | |

*EtOH is eliminated during granulate desiccation

### Dissolution Tests

Each of the foregoing diphenhydramine HCl formulations was tested according to the protocol described below, and the time to reach a release higher than 85% was recorded.
Analytical Equipment: UV
Apparatus: Paddle (50 rpm)
Mediums: pH 1.2/pH 4.5/pH 6.8 (prepared according to the USP)
Wavelength: 265 nm
Volume: 400 ml
Withdrawal times: 2.5, 5, 10, and 15 minutes

| | Time (min) for release > 85% (Average of 4 units) | | |
|---|---|---|---|
| Combinations | Medium pH 1.2 | Medium pH 4.5 | Medium pH 6.8 |
| A | 2.5 | 2.5 | 2.5 |
| B | 2.5 | 5 | 2.5 |
| C | 2.5-5 | 2.5 | 2.5 |
| D | 2.5-5 | 2.5 | 2.5 |
| E | 5 | 2.5 | 2.5-5 |
| F | 2.5 | 2.5 | 2.5 |

### Example 1c

### Dextromethorphan HBr

### Combinations

| | A | B | C | D | E | F |
|---|---|---|---|---|---|---|
| Dextro- | 10.0 g | 10.0 g | 10.0 g | 10.0 g | 10.0 g | 10.0 g |
| methorphan | | | | | | |
| HBr | | | | | | |
| Mannitol/ | 37.5 g | 37.5 g | 37.5 g | 37.5 g | 37.5 g | 37.5 g |
| maize starch | | | | | | |
| EtOH (90%) | — | 6.0 g | — | — | — | — |
| Solution at | — | — | 8.5 g | — | — | — |
| 10% of | | | | | | |
| HPMCP in | | | | | | |
| EtOH (90%)* | | | | | | |
| Solution at | — | — | — | 8.5 g | — | — |
| 10% of | | | | | | |
| KLUCEL ® in | | | | | | |
| EtOH (90%)* | | | | | | |
| Solution at | — | — | — | — | 8.5 g | — |
| 10% of | | | | | | |
| ETHOCEL ® | | | | | | |
| in EtOH | | | | | | |
| (90%)* | | | | | | |
| Solution at | — | — | — | — | — | 8.5 g |
| 10% of | | | | | | |
| EUDRAGIT ® | | | | | | |
| in EtOH | | | | | | |
| (90%)* | | | | | | |

*EtOH is eliminated during granulate desiccation

### Dissolution Tests

Each of the foregoing dextromethorphan HBr formulations was tested according to the protocol described below, and the time to reach a release higher than 85% was recorded.
Analytical Equipment: UV
Apparatus: Paddle (50 rpm)
Mediums: pH 1.2/pH 4.5/pH 6.8 (prepared according to the USP)
Wavelength: 280 nm
Volume: 400 ml
Withdrawal times: 2.5, 5, 10, and 15 minutes

| | Time (min) for release > 85% (Average of 4 units) | | |
|---|---|---|---|
| Combinations | Medium pH 1.2 | Medium pH 4.5 | Medium pH 6.8 |
| A | 2.5 | 2.5 | 2.5 |
| B | 2.5 | 2.5 | 2.5 |
| C | 2.5 | 2.5 | 2.5 |
| D | 2.5 | 2.5 | 2.5 |
| E | 2.5 | 2.5 | 2.5 |
| F | 2.5 | 2.5 | 2.5 |

### Example 1d

### Optimization of HPMCP Content in Dextromethorphan HBr Formulation

For the prototype containing dextromethorphan HBr and coated with HPMCP (compare combination “C” in Example 1b), an additional prototype (identified as prototype “G”) was developed containing a total amount of 12.75 g of solution at 10% of HPMCP.

| | G |
|---|---|
| Dextromethorphan HBr | 10.0 g |
| Mannitol/maize starch | 37.5 g |
| Solution at 10% of HPMCP in EtOH (90%)* | 12.75 g |

*EtOH is eliminated during granulate desiccation

Dissolution was tested using the following conditions, with the results of such testing reported below.
Analytical Equipment: UV
Apparatus: Paddle (50 rpm)
Mediums: pH 1.2/pH 4.5/pH 6.8 (prepared according to the USP)
Wavelength: 280 nm
Volume: 400 ml
Withdrawal times: 2.5, 5, 10, and 15 minutes

| | Time (min) for release > 85% (Average of 4 units) | | |
|---|---|---|---|
| Combinations | Medium pH 1.2 | Medium pH 4.5 | Medium pH 6.8 |
| G | 2.5 | 5-10 | 5-10 |

The foregoing prototypes demonstrated that practically all coating agents yielded the same dissolution profile, and that an increase in the coating agent quantity unexpectedly improves the palatability of the formulation without adversely affecting the dissolution profile in a significant way.

### Example 2

### Single Active Ingredient Formulations

Based on the results obtained from the initial prototypes in Example 1, formulations of different drugs were prepared in order to evaluate the physical performance of different combinations of coating agent and active ingredients. The granules and the corresponding formulations are described in the following paragraphs.

All batches were manufactured according to the following general procedure:

### Manufacturing Equipment

Balances of various types;
High share mixer: Diosna Laboratory mixer P1/6;
Sieves;
Static oven;
Mixing machine

### Granulate Preparation

The granule components were separately weighed. The granulating solution was then prepared by dissolving the coating agent and any sweeteners and flavoring agents in a prescribed volume of ethanol. The active ingredient (passed through an appropriate sieve if aggregates were present) was introduced to a high share mixer (Diosna). The mixer was turned on, and the granulating solution was slowly added; when all the granulating solution was added, the mixer speed was increased, the chopper on the high shear mixer turned on, and the PEARLITOL FLASH® slowly added. The granulation continued for about 5 minutes and the resulting granulate was then passed through a 630μ stainless steel sieve. Examples 2a and 2c were prepared in this manner.

In some cases the mannitol/maize starch was added first to the high shear mixer. The active ingredient was dissolved in the granulating solution, and the granulating solution was added to the mannitol/maize starch while the mixer was on. Once again, after the granulating solution was added, the mixer speed was increased and the Diosna's chopper turned on. After about 5 minutes the resulting granulates was then passed through a 630μ stainless steel sieve. Example 2b was prepared in this manner.

After the granular mixture was prepared, it was put into a static oven at 30° C. for about 6 hours, and passed through a 630μ stainless steel sieve.

### Final Mix Preparation

Any extra granules were separately weighed, mixed with the active ingredient granules, and mixed for 5 minutes.

### Example 2a

### Ibuprofen

Granulates of ibuprofen were prepared according to the quali-quantitative formulations reported in the table below:

| | bkT027/ | bkT027/ | bkT027/ | bkT027/ | bkT032/63 |
|---|---|---|---|---|---|
| | 5 | 132 | 131 | 130 | granulate |
| Ibuprofen | 200.0 g | 200.0 g | 200.0 g | 200.0 g | 200.0 g |
| Mannitol/maize | 380.0 g | 380.0 g | 380.0 g | 380.0 g | .80.0 g |
| starch | | | | | |
| Sucralose | 7.0 g | 7.0 g | 7.0 g | 7.0 g | 12.0 g |
| Mint Liquid Flavor | 2.0 g | 2.0 g | 2.0 g | 2.0 g | — |
| Solution at 10% | 100.0 g | — | — | — | |
| of HPMCP | | | | | |
| in EtOH (90%)* | | | | | |
| Solution at 10% | — | 100.0 g | — | — | |
| of KLUCEL ® | | | | | |
| in EtOH (90%)* | | | | | |
| Solution at 10% | — | — | 100.0 g | — | |
| of ETHOCEL ® | | | | | |
| in EtOH (90%)* | | | | | |
| Solution at 10% | — | — | — | 100.0 g | |
| of EUDRAGIT ® | | | | | |
| in EtOH (90%)* | | | | | |
| Solution at 13.3% | | — | — | — | 35.0 g |
| of HPMCP in | | | | | |
| EtOH:Acetone | | | | | |
| 45:20* | | | | | |

*Solvents are eliminated during granulate desiccation

Details of the MAA/MMA and HPMCP formulations are reported below.

### Qualitative and Quantitative Formulation Batch bkT027/133 (with MAA/MMA)

| | mg/dose | |
|---|---|---|
| Component | bkT027/133 | Function |
| Granule | | |
| Ibuprofen | 200.0 | Active Ingredient |
| Sucralose | 7.0 | Sweetener |
| PEARLITOL | 380.0 | Bulking agent with |
| FLASH ® | | superdisintegrant |
| Mint liquid | 2.0 | Flavor |
| Flavor | | |
| Solution at 10% | 100.0 | Granulating solution |
| of EUDRAGIT ® | | |
| in EtOH (90%)* | | |
| Extragranules | | |
| Sucralose | 8.0 | Sweetener |
| PEARLITOL | 364.0 | Bulking agent with |
| FLASH ® | | superdisintegrant |
| Raspberry Flavor | 18.0 | Flavor |
| Anise Flavor | 10.0 | Flavor |

*EtOH is eliminated during granulate desiccation

### Qualitative and Quantitative Formulation Batch bkT027/15 (with HPMCP)

| | mg/dose | |
|---|---|---|
| Component | bkT027/15 | Function |
| Ibuprofen | 200.0 | Active Ingredient |
| Sucralose | 7.0 | Sweetener |
| PEARLITOL | 380.0 | Bulking agent with |
| FLASH ® | | superdisintegrant |
| Mint liquid | 2.0 | Flavor |
| Flavor | | |
| Solution at 10% | 100.0 | Granulating solution |
| of HPMCP in | | |
| EtOH (90%)* | | |
| Extragranules | | |
| Sucralose | 4.0 | Sweetener |
| PEARLITOL | 280.0 | Bulking agent with |
| FLASH ® | | superdisintegrant |
| Raspberry Flavor | 18.0 | Flavor |
| Anise Flavor | 8.2 | Flavor |

*EtOH is eliminated during granulate desiccation

### Qualitative and Quantitative Formulation Batch bkT032/63 (with HPMCP)

| | mg/dose | |
|---|---|---|
| | bkT032/63 | |
| | finished | |
| Component | product | Function |
| Granule | | |
| Ibuprofen | 200.0 | Active Ingredient |
| Sucralose | 12.0 | Sweetener |
| PEARLITOL | 80.0 | Bulking agent with |
| FLASH ® | | superdisintegrant |
| Solution at | 35.0 | Granulating solution |
| 13.3% of HPMCP | | |
| in EtOH:Acetone | | |
| 45:20* | | |
| Extragranules | | |
| Acid Citric | 40.0 | Flavor Enhancer |
| Anhydrous | | |
| Sodium Chloride | 20.0 | Flavor Enhancer |
| Sucralose | 10.0 | Sweetener |
| PEARLITOL | 398.0 | Bulking agent with |
| FLASH ® | | superdisintegrant |
| Banana Flavor | 24.0 | Flavor |
| Mint Flavor | 16.0 | Flavor |

*Solvents are eliminated during granulate desiccation

### Tests on the Final Mix

### Taste

The taste evaluations showed that batches bkT027/133 and bkT032/63 containing respectively MAA/MMA and HPMCP were the most palatable.

### Dissolution Test

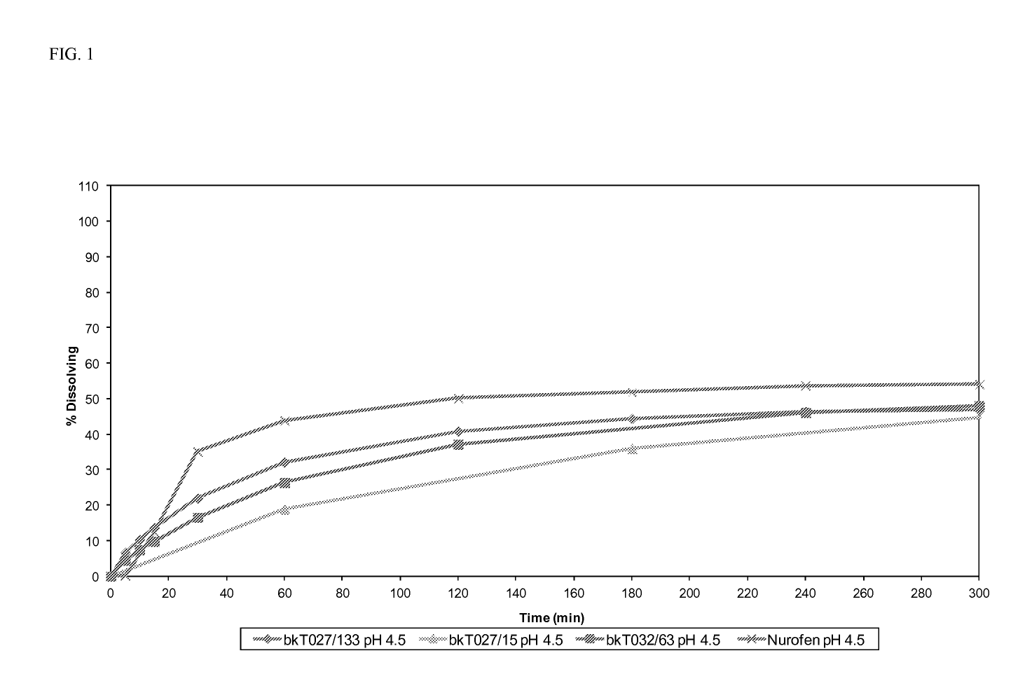

The dissolution profile of batches bkT027/133, bkT027/15 and bkT032/63 were evaluated according to the following analytical conditions:
Analytical Equipment: HPLC
Apparatus: Paddle (50 rpm)
Medium: Acetate Buffer pH 4.5 (prepared according to USP)
Volume: 900 ml
Withdrawal times: 5, 10, 15, 30, 60, 120, 240, 300, and 360 minutes
   The dissolution profiles obtained on the APR's prototypes are reported in comparison with the dissolution profile of the Nurofen 200 mg as a reference market product in FIG. 1.

### Considerations on the Results

Coating agents HPMCP and MAA/MMA gave similar dissolution profiles; the presence of PEARLITOL FLASH® in the formulation allow in any case the release of the API even though the pH of the medium is not able to solubilize the coating layer. The dissolution profiles of the prototypes have a trend similar to the ones of the reference market product (Nurofen 200 mg).

### Example 2b

### Loratadine HCl

An oral dispersible formulation containing loratadine HCl was prepared both with and without HPMCP in the granular mixture. Each of the formulations was tested with and without the inclusion of flavoring agents included in the extra granules to determine the dissolution properties of the finished formulation using the UV method. The UV method is incompatible with flavors due to typical optical interferences.

### Qualitative and Quantitative Formulation Batch bkT027/49 and bkT027/51 (without HPMCP)

| | mg/dose | mg/dose | |
|---|---|---|---|
| Component | bkT027/49 | bkT027/51 | Function |
| Granule | | | |
| Loratadine HCl | 10.0 | 10.0 | Active Ingredient |
| Sucralose | 10.0 | 10.0 | Sweetener |
| PEARLITOL | 200.0 | 200.0 | Bulking agent with |
| FLASH ® | | | superdisintegrant |
| EtOH (90%)* | 40.0 | 40.0 | Granulating solution |
| Extra Granules | | | |
| Sucralose | 5.0 | 5.0 | Sweetener |
| PEARLITOL | 140.0 | 140.0 | Bulking agent with |
| FLASH ® | | | superdisintegrant |
| Orange Flavor | 20.0 | — | Flavor |
| Peach Flavor | 15.0 | — | Flavor |

*EtOH is eliminated during granulate desiccation

### Qualitative and Quantitative Formulation Batch bkT027/53 and bkT027/55 (with HPMCP)

| | mg/dose | mg/dose | |
|---|---|---|---|
| Component | bkT027/53 | bkT027/55 | Function |
| Granule | | | |
| Loratadine HCl | 10.0 | 10.0 | Active Ingredient |
| Sucralose | 10.0 | 10.0 | Sweetener |
| PEARLITOL | 200.0 | 200.0 | Bulking agent with |
| FLASH ® | | | superdisintegrant |
| Solution at 10% | 40.0 | 40.0 | Granulating solution |
| of HPMCP in | | | |
| EtOH (90%)* | | | |
| Extra Granules | | | |
| Sucralose | 5.0 | 5.0 | Sweetener |
| PEARLITOL | 136.0 | 136.0 | Bulking agent with |
| FLASH ® | | | superdisintegrant |
| Orange Flavor | 20.0 | — | Flavor |
| Peach Flavor | 15.0 | — | Flavor |

*EtOH is eliminated during granulate desiccation

### Tests on the Final Mix and on Granulates

### Taste

The prototypes batches bkT023/49 and bkT027/53 (the prototypes with and without HPMCP, both with flavors) were tested: due to the neutral flavor characteristic of the drug, both the prototypes were palatable.

### Dissolution Test

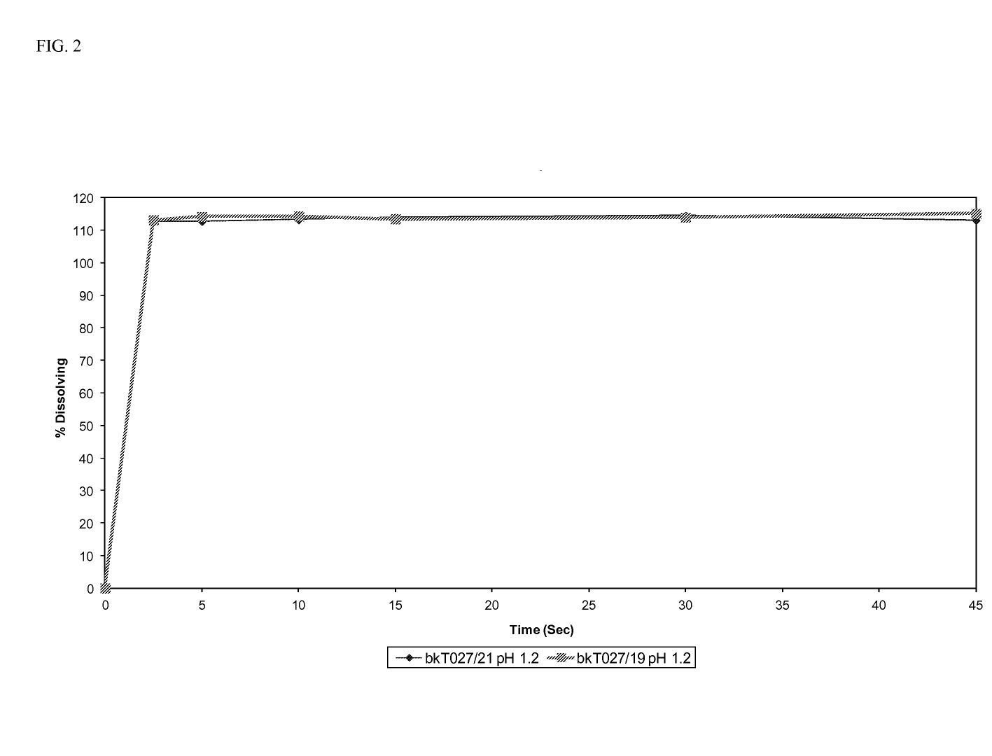

Dissolution profiles were generated on granulate batches bkT027/19 (contained in finished product batch bkT027/51) and bkT027/21 (contained in finished product batch bkT027/55), which are the prototypes without and with HPMCP (both without the extra granules). Dissolution testing was performed according to the following analytical conditions:
Analytical Equipment: UV
Apparatus: Paddle (50 rpm)
Medium: Buffer pH 1.2 (prepared according to USP method)
Wavelength: 280 nm
Volume: 900 ml
Withdrawal times: 2.5, 5, 10, 15, 30, and 45 minutes
   The dissolution profiles for the two batches are presented in FIG. 2.

As can be seen, the coating agent HPMCP had no effect on the dissolution of loratadine from this formulation.

### Example 2c

### Ketoprofene Lysine Salt: granulation with water

### Qualitative and Quantitative Formulation Batch bkT032/55

| Component | mg/dose | Function |
|---|---|---|
| Granules | | |
| Ketprofene Lysine Salt | 80.0 | Active Ingredient |
| Na Cl grinded | 10.0 | Flavor enhancers |
| PEARLITOL | 10.0 | Bulking agent with |
| FLASH ® | | super disintegrant |
| Solution at 30% of | 1.98 | Granulating solution |
| EUDRAGIT L30 D55 | | |
| in water * | | |
| Extra granules | | |
| Sucralose | 15.0 | Sweetener |
| Xylitol | 540.0 | Bulking agent |
| PEARLITOL | 805.0 | Bulking agent with |
| FLASH ® | | super disintegrant |
| Mint | 3.0 | Flavor |
| KHCO3 | 35.0 | Flavor enhancer |

* Water is eliminated during granulate desiccation

### Dissolution Tests

The formulation containing Ketoprofene Lysine Salt was tested according to the analytical conditions described below, and the dissolution profile was determined.
Analytical Equipment: HPLC
Apparatus: Paddle (50 rpm)
Medium: Acetate Buffer pH 4.5 (prepared according to USP)
Volume: 900 ml
Withdrawal times: 2.5, 5, 10, 15, and 30 minutes

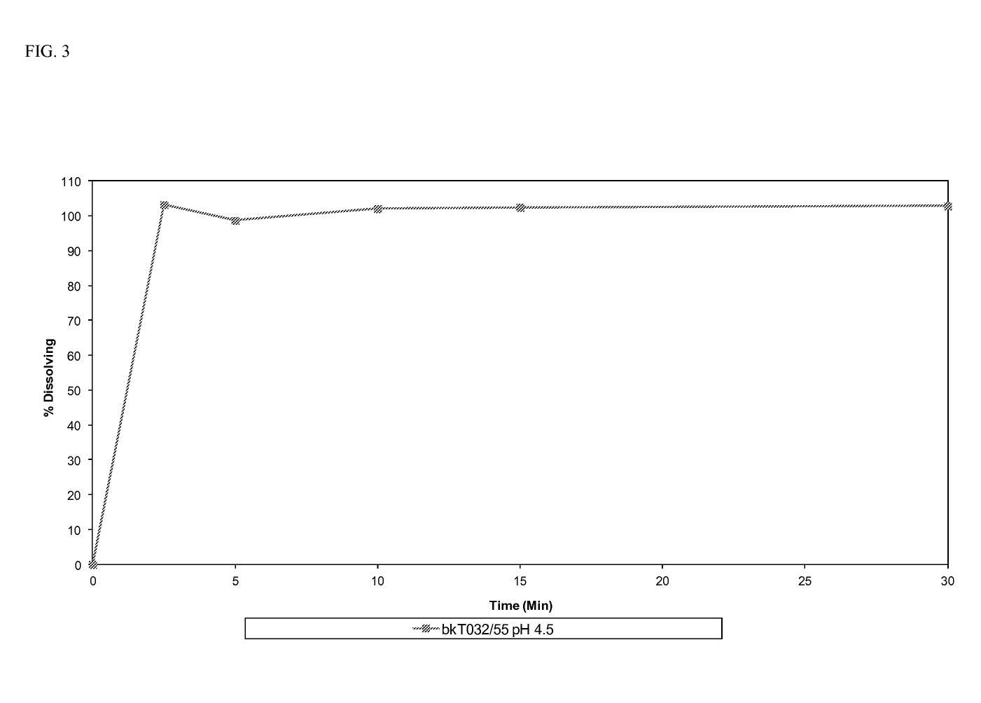

The dissolution profile obtained on the APR's prototype is reported in FIG. 3:

The foregoing prototype demonstrated that a water soluble coating agent yielded good palatability without delaying the dissolution profile. The dissolution profile obtained is typical of an immediate release oral formulation.

### Example 3

### Formulations Containing Multiple Active Ingredients

Prototype formulations containing multiple active ingredients were also prepared according to the general manufacturing methods described in Example 2.

### Example 3a

### Acetaminophen 250 mg, Guaifenesin 200 mg, Dextromethorphan HBr 10 mg, Phenylephrine HCl 5 mg

### Selection of the Granulating Agent

Separate granulates of Acetaminophen and Guaifenesin or Dextromethorphan HBr and Phenylephrine HCl were prepared according to the quali-quantitative formulations reported below

### Granulate A

| | bkT027/ | bkT027/ | bkT027/ | bkT027/ |
|---|---|---|---|---|
| | 95 | 117 | 116 | 115 |
| Acetaminophen | 325.0 g | 325.0 g | 325.0 g | 325.0 g |
| Guaifenesin | 200.0 g | 200.0 g | 200.0 g | 200.0 g |
| Sucralose | 20.0 | 20.0 | 20.0 | 20.0 |
| Mannitol/maize | 250.0 g | 250.0 g | 250.0 g | 250.0 g |
| starch | | | | |
| Solution at 20% | 80.0 g | — | — | — |
| of HPMCP in | | | | |
| EtOH:H2O:Acetone = | | | | |
| 75:5:20* | | | | |
| Solution at 20% | — | 80.0 | — | — |
| of KLUCEL ® | | | | |
| in EtOH (90%)* | | | | |
| Solution at 20% | — | — | 80.0 | — |
| of ETHOCEL ® | | | | |
| in EtOH (90%)* | | | | |
| Solution at 20% | — | — | — | 80.0 g |
| of MAA/MMA | | | | |
| in EtOH (90%)* | | | | |

*EtOH is eliminated during granulate desiccation

### Granulate B

| | bkT027/ | bkT027/ | bkT027/ | bkT027/ |
|---|---|---|---|---|
| | 106 | 120 | 118 | 119 |
| Dextrome- | 10.0 g | 325.0 g | 325.0 g | 325.0 g |
| thorphan HBr | | | | |
| Phenylephrine | 5.0 g | 200.0 g | 200.0 g | 200.0 g |
| HCl | | | | |
| Mannitol/maize | 364.0.0 g | 364.0.0 g | 364.0.0 g | 364.0.0 g |
| starch | | | | |
| Sucralose | 8.0 g | 8.0 g | 8.0 g | 8.0 g |
| Solution | 70.0 g | — | — | — |
| at 20% of | | | | |
| HPMCP in | | | | |
| EtOH (90%)* | | | | |
| Solution | — | 70.0 g | — | — |
| at 20% of | | | | |
| KLUCEL ® in | | | | |
| EtOH (90%)* | | | | |
| Solution | — | — | 70.0 g | — |
| at 20% of | | | | |
| ETHOCEL ® | | | | |
| in EtOH | | | | |
| (90%)* | | | | |
| Solution | — | — | — | 70.0 g |
| at 20% of | | | | |
| EUDRAGIT ® | | | | |
| in EtOH | | | | |
| (90%)* | | | | |

*EtOH is eliminated during granulate desiccation

Batch bkT027/115 (granulate A with MAA/MMA) and batch bkT027/119 (granulate B with MAA/MMA) were determined to be the most palatable.

Additional batches bkT027/95 (granulate A with HPMCP) and bkT027/106 (granulate B with HPMCP) were also prepared for comparison.

### Qualitative and Quantitative Formulation Batch bkT027/111

| Component | mg/dose | Function |
|---|---|---|
| Granulate A | | |
| Acetaminophen | 325.0 | Active Ingredient |
| Guaifenesin | 200.0 | Active Ingredient |
| Sucralose | 20.0 | Sweetener |
| PEARLITOL | 250.0 | Bulking agent with |
| FLASH ® | | superdisintegrant |
| Solution at 20% of | 80.0 | Granulating solution |
| HPMCP:H2O:Acetone = | | |
| 75:5:20* | | |
| Granulate B | | |
| Dextromethorphan | 10.0 | Active Ingredient |
| HBr | | |
| Phenylephrine HCl | 5.0 | Active Ingredient |
| Sucralose | 8.0 | Sweetener |
| PEARLITOL | 364.0 | Bulking agent with |
| FLASH ® | | superdisintegrant |
| Solution at 20% of | 70.0 | Granulating solution |
| HPMCP in EtOH (90%)* | | |
| Extra granules | | |
| Sucralose | 25.0 | Sweetener |
| PEARLITOL | 103.0 | Bulking agent with |
| FLASH ® | | superdisintegrant |
| Xylitol | 200.0 | Bulking agent |
| Peach Flavor | 15.0 | Flavor |
| Raspberry Flavor | 45.0 | Flavor |

*EtOH is eliminated during granulate desiccation

### Qualitative and Quantitative formulation batch bkT027/124

| Component | mg/dose | Function |
|---|---|---|
| Granulate A | | |
| Acetaminophen | 325.0 | Active Ingredient |
| Guaifenesin | 200.0 | Active Ingredient |
| Sucralose | 20.0 | Sweetener |
| PEARLITOL | 250.0 | Bulking agent with |
| FLASH ® | | superdisintegrant |
| Solution at 20% of | 80.0 | Granulating solution |
| EUDRAGIT ® in EtOH | | |
| (90%)* | | |
| Granulate B | | |
| Dextromethorphan | 10.0 | Active Ingredient |
| HBr | | |
| Phenylephrine HCl | 5.0 | Active Ingredient |
| Sucralose | 8.0 | Sweetener |
| PEARLITOL | 364.0 | Bulking agent with |
| FLASH ® | | superdisintegrant |
| Solution at 20% of | 70.0 | Granulating solution |
| EUDRAGIT ® in EtOH | | |
| (90%)* | | |
| Extra granules | | |
| Sucralose | 25.0 | Sweetener |
| PEARLITOL | 103.0 | Bulking agent with |
| FLASH ® | | superdisintegrant |
| Xylitol | 200.0 | Bulking agent |
| Peach Flavor | 15.0 | Flavor |
| Raspberry Flavor | 45.0 | Flavor |

*EtOH is eliminated during granulate desiccation

### Tests on the Final Mix

### Taste

All formulations were palatable and could be taken without water, but prototype bkT027/124 resulted in the best taste.

### Dissolution Test

The dissolution profile of batches bkT027/124 was carried out in an acidic medium (pH 1.2) according to the following analytical conditions:

### Acetaminophen and Guaifenesin

Analytical Equipment: UV and HPLC
Apparatus: Paddle (50 rpm)
Medium: pH 1.2 (prepared according to the USP)
Column: Alltima C18 150×4.6 mm or equivalent
Wavelength: 275 nm
Inj. Volume: 10 μl
Temperature of the column: 45° C.
Mobile Phase: H2O:MeOH:CH3COOH=69:28:3
Flow: 1.5 ml/min
Volume: 400 ml
Withdrawal times: 2.5, 5, 10, 15, 30, 60, 120, and 180 minutes

### Phenylephrine HCl

Analytical Equipment: HPLC
Apparatus: Paddle (50 rpm)
Medium: pH 1.2 (prepared according to the USP)
Column: Alltima C18 250×4.6 mm or equivalent
Wavelength: 280 nm
Inj. Volume: 20 μl
Temperature of the column: 25° C.
Mobile Phase: H2O:MeOH:1-Octanesulphonic Acid=49.89:50:0.11 (correct to pH3.00±0.10 with Phosphoric Acid)
Flow: 1.0 ml/min
Volume: 400 ml
Withdrawal times: 2.5, 5, 10, 15, 30, 60, 120, and 180 minutes

### Dextromethorphan HBr

Analytical Equipment: HPLC
Apparatus: Paddle (50 rpm)
Medium: pH 1.2 (prepared according to the USP)
Column: Simmetry C18 250×4.6 mm or equivalent
Wavelength: 280 nm
Inj. Volume: 10 μl
Temperature of the column: 25° C.
Mobile Phase: (3.11 g/L Na Docusate+0.56 g/L of Ammonium Nitrate in

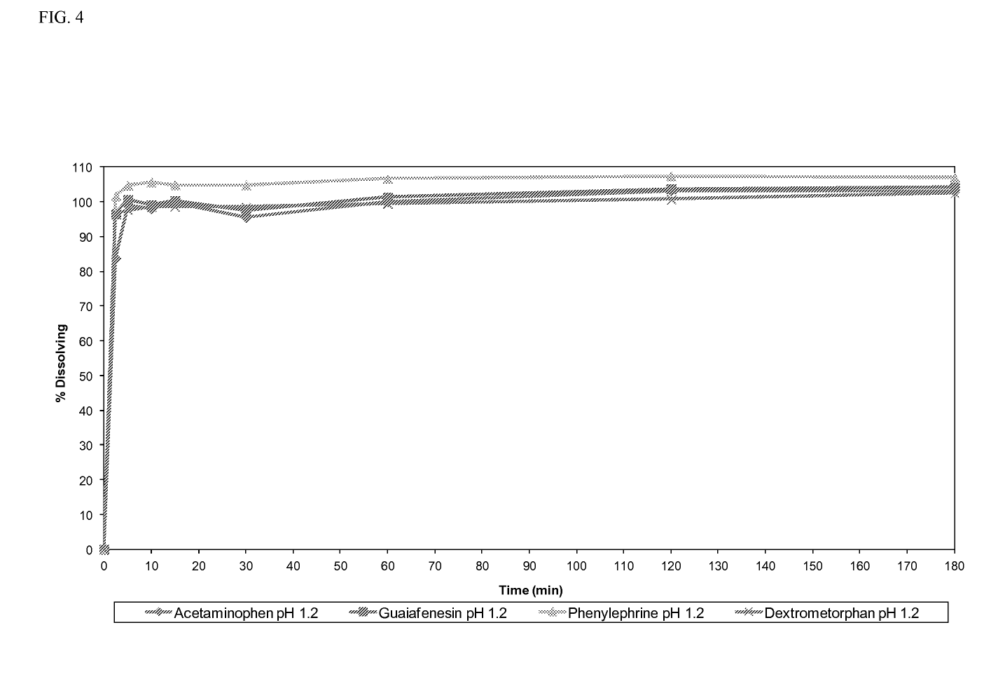

Water): CH3CN=30:70
Flow: 1.0 ml/min
Volume: 400 ml
Withdrawal times: 2.5, 5, 10, 15, 30, 60, 120, and 180 minutes
   The dissolution profile obtained on 6 units of batch bkT027/124 at pH 1.2 is reported in FIG. 4.

### Example 3b

### Acetaminophen 250 mg, Acetylsalicylic Acid (ASA) 25 mg, Caffeine 65 mg

### Selection of the Granulating Agent

Granulates containing both acetaminophen and ASA were prepared according to the quali-quantitative formulations reported below:

### Granulate A

| | bkT027/ | bkT027/ | bkT027/ | bkT027/ |
|---|---|---|---|---|
| | 103 | 123 | 121 | 122 |
| Acetaminophen | 250.0 g | 250.0 g | 250.0 g | 250.0 g |
| ASA | 250.0 g | 250.0 g | 250.0 g | 250.0 g |
| Sucralose | 7.0 g | 7.0 g | 7.0 g | 7.0 g |
| Mannitol/maize | 180.0 g | 180.0 g | 180.0 g | 180.0 g |
| starch | | | | |
| Solution at 10% | 60.0 g | — | — | — |
| of HPMCP in | | | | |
| EtOH (90%)* | | | | |
| Solution at 10% | — | 60.0 g | — | — |
| of KLUCEL ® | | | | |
| in EtOH (90%)* | | | | |
| Solution at 10% | — | — | 60.0 g | — |
| of ETHOCEL ® | | | | |
| in EtOH (90%)* | | | | |
| Solution at 10% | — | — | — | 60.0 g |
| of EUDRAGIT ® | | | | |
| in EtOH (90%)* | | | | |

*EtOH is eliminated during granulate desiccation

Taste evaluations showed that batch bkT027/122 (granulate A with MAA/MMA) was the most palatable, and this formulation was used in the final formulation. In addition, another final formulation containing HPMCP for a comparison was prepared with batch bkT027/103 (granulate A with HPMCP).

Due to the bitter taste of caffeine, this active ingredient was also coated according to the quali-quantitative formulation reported below:

Coated Caffeine (Batch bkT027/112)

| Component | mg/dose | Function |
|---|---|---|
| Caffeine | 65.000 | Active Ingredient |
| Cellulose PH 101 | 45.500 | Bulking agent |
| Sucralose | 2.275 | Sweetener |
| Solution at 22.5% of | 70.68 | Granulating solution |
| HPMCP, Methocel and | | and Plasticizing agent |
| Stearic Acid in EtOH | | |
| (90%)* Ratio | | |
| HPMCP:Methocel:Stearic | | |
| Acid = 1:6:2 | | |

*EtOH is eliminated during granulate desiccation

### Manufacturing Method

A different manufacturing method was used to prepare the coated caffeine. The coating solution was prepared by dissolving both the polymers (HPMCP and Methocel) in EtOH at 90% at 55° C., but all of the other steps remained the same.

The final oral dispersible formulations containing Acetaminophen, Acetyl Salicylic Acid and Caffeine was prepared according to the following quali/quantitative formulation:

### Qualitative and Quantitative Formulation Batch bkT027/128

| Component | mg/dose | Function |
|---|---|---|
| Granulate A | | |
| Acetaminophen | 250.0 | Active Ingredient |
| Acetyl Salicylic Acid | 250.0 | Active Ingredient |
| Sucralose | 7.0 | Sweetener |
| PEARLITOL | 180.0 | Bulking agent with |
| FLASH ® | | superdisintegrant |
| Solution at 10% of | 60.0 | Granulating solution |
| EUDRAGIT ® in EtOH | | |
| (90%)* | | |
| Granulate B | | |
| Coated Caffeine | 128.0 | Active Ingredient |
| (corresponding to 50.8%) | | |
| Extra granules | | |
| Sucralose | 15.0 | Sweetener |
| PEARLITOL | 584.0 | Bulking agent with |
| FLASH ® | | superdisintegrant |
| Peach Flavor | 20.0 | Flavor |
| Strawberry Flavor | 60.0 | Flavor |

*EtOH is eliminated during granulate desiccation

### Tests on the Final Mix

### Taste

Volunteers tested the prototype batch bkT027/128, and despite the high concentration of different non-palatable active ingredients, the formulations were palatable and could be taken without water.

### Dissolution Test

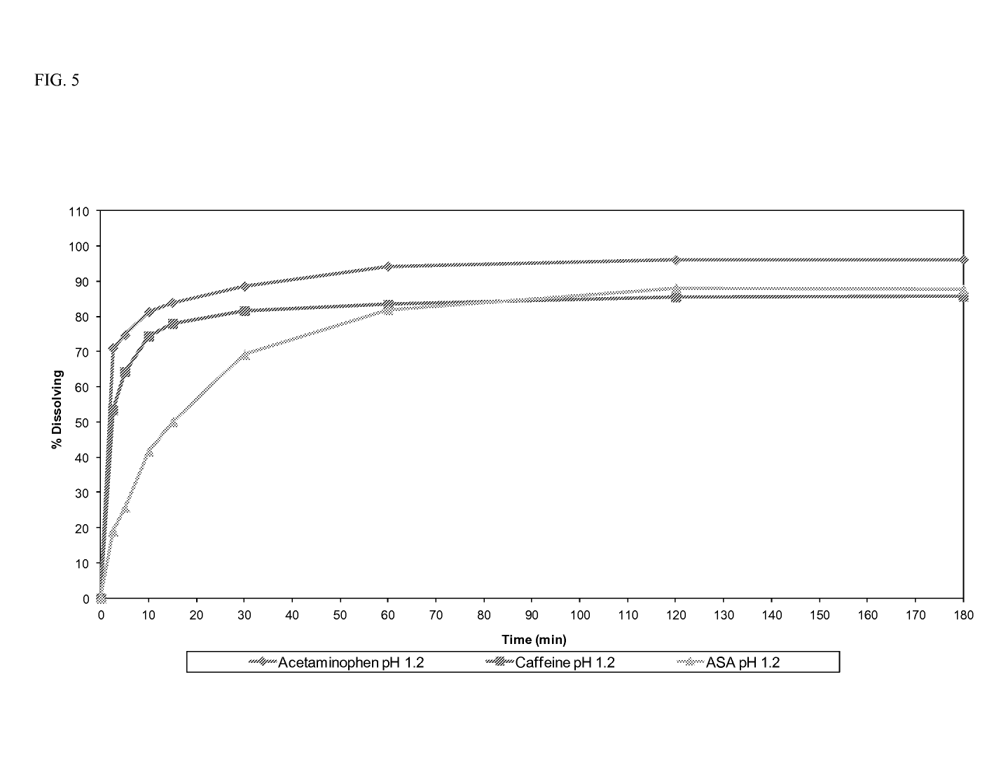

The dissolution profile of batch bkT027/128 was carried out in pH 1.2 medium according to the following analytical conditions:
Analytical Equipment: HPLC
Apparatus: Paddle (50 rpm)
Medium: pH 1.2 (prepared according to the USP)
Column: Alltima C18 150×4.6 mm or equivalent
Wavelength: 275 nm
Inj. Volume: 10 μl
Temperature of the column: 45° C.
Mobile Phase: H2O:MeOH:CH3COOH=69:28:3
Flow: 1.5 ml/min
Volume: 900 ml
Withdrawal times: 2.5, 5, 10, 15, 30, 60, 120, and 180 minutes
   The dissolution profile of batch bkT027/128 was generated at pH 1.2 according to the analytical conditions reported above, and is reported in FIG. 5.

### Example 3c

### Diphenhydramine HCl 25 mg/Acetaminophen 500 mg

### Selection of the Granulating Agent

Several granulates were prepared according to the quali-quantitative formulations reported below:

### Granulate A

| | bkT027/ | bkT027/ | bkT027/ | bkT027/ |
|---|---|---|---|---|
| | 102 | 127 | 126 | 125 |
| Acetaminophen | 500.0 g | 500.0 g | 500.0 g | 500.0 g |
| Diphenhydramine HCl | 25.0 g | 25.0 g | 25.0 g | 25.0 g |
| Sucralose | 25.0 g | 25.0 g | 25.0 g | 25.0 g |
| Mannitol/maize starch | 303.0 g | 303.0 g | 303.0 g | 303.0 g |
| Solution at 10% of | 80.0 g | — | — | — |
| HPMCP in EtOH (90%)* | | | | |
| Solution at 10% of | — | 80.0 g | — | — |
| KLUCEL ® in | | | | |
| EtOH (90%)* | | | | |
| Solution at 10% of | — | — | 80.0 g | — |
| ETHOCEL ® in | | | | |
| EtOH (90%)* | | | | |
| Solution at 10% of | — | — | — | 80.0 g |
| EUDRAGIT ® in | | | | |
| EtOH (90%)* | | | | |

*EtOH is eliminated during granulate desiccation

The final oral dispersible formulation was prepared with granulates bkT027/125 (granulate A with MAA/MMA); in addition another formulation containing the granulates bkT027/102 (granulate A with HPMCP) was prepared for a comparison. Taste evaluations showed that batch bkT027/125 (granulate A with MAA/MMA) was the most palatable.

### Qualitative and Quantitative Formulation Batch bkT027/108

| Component | mg/dose | Function |
|---|---|---|
| Granulate A | | |
| Diphenhydramine HCl | 25.0 | Active Ingredient |
| Acetaminophen | 500.0 | Active Ingredient |
| Sucralose | 25.0 | Sweetener |
| PEARLITOL | 303.0 | Bulking agent with |
| FLASH ® | | superdisintegrant |
| Solution at 10% of | 80.0 | Granulating solution |
| HPMCP in EtOH (90%)* | | |
| Extra granules | | |
| Sucralose | 10.0 | Sweetener |
| PEARLITOL | 239.0 | Bulking agent with |
| FLASH ® | | superdisintegrant |
| Peach | 30.0 | Flavor |
| Strawberry | 60.0 | Flavor |

*EtOH is eliminated during granulate desiccation

### Qualitative and Quantitative Formulation Batch bkT027/129

| Component | mg/dose | Function |
|---|---|---|
| Granulate A | | |
| Diphenhydramine HCl | 25.0 | Active Ingredient |
| Acetaminophen | 500.0 | Active Ingredient |
| Sucralose | 25.0 | Sweetener |
| PEARLITOL | 303.0 | Bulking agent with |
| FLASH ® | | superdisintegrant |
| Solution at 10% of | 80.0 | Granulating solution |
| EUDRAGIT in EtOH | | |
| (90%)* | | |
| Extra granules | | |
| Sucralose | 10.0 | Sweetener |
| PEARLITOL | 239.0 | Bulking agent with |
| FLASH ® | | superdisintegrant |
| Peach | 30.0 | Flavor |
| Strawberry | 60.0 | Flavor |

*EtOH is eliminated during granulate desiccation

### Tests on the Final Mix

### Taste

Volunteers tested the prototype batch bkT027/129 and, despite the high concentration of different non-palatable active ingredients, the formulation was palatable and could be taken without water.

### Dissolution Test

The dissolution profile of batch bkT027/108 was carried out in three different medium (pH 1.2, 4.5 and 6.8) and batch bkT027/129 at pH 1.2 according to the following analytical conditions reported in the USP product monographs:

### Acetaminophen

Analytical Equipment: HPLC
Apparatus: Paddle (50 rpm)
Medium: pH 1.2/4.5/6.8 (prepared according to the USP)
Column: Alltima C18 150×4.6 mm or equivalent
Wavelength: 275 nm
Inj. Volume: 10 μl
Temperature of the column: 45° C.
Mobile Phase: H2O:MeOH:CH3COOH=69:28:3
Flow: 1.5 ml/min
Volume: 400 ml
Withdrawal times: 2.5, 5, 10, 15, 30 (60, and 120 minutes for bkT027/129)

### Diphenhydramine HCl

Analytical Equipment: HPLC
Apparatus: Paddle (50 rpm)
Medium: pH 1.2/4.5/6.8 (prepared according to the USP)
Column: Luna CN 5μ 100A 150*4.6 mm or equivalent
Wavelength: 228 nm
Inj. Volume: 10 μl
Temperature of the column: 25° C.
Mobile Phase: H2O:MeOH: TEA=49.5:50.0:0.5
Flow: 1.0 ml/min
Volume: 400 ml
Withdrawal times: 2.5, 5, 10, 15, 30, (60, and 120 for bkT027/129)

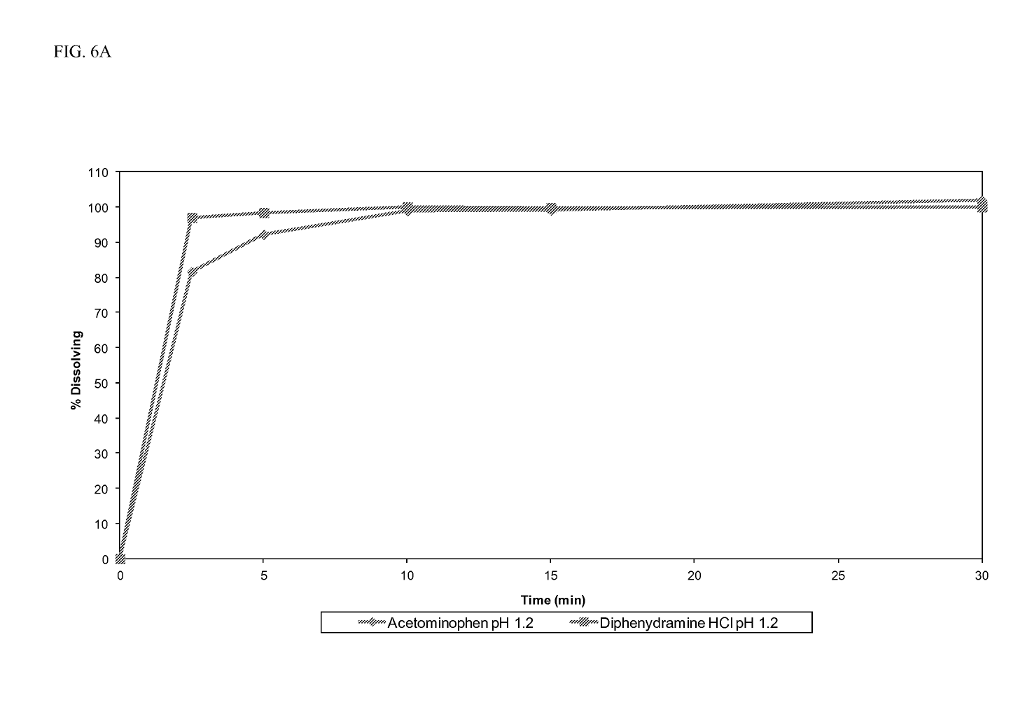

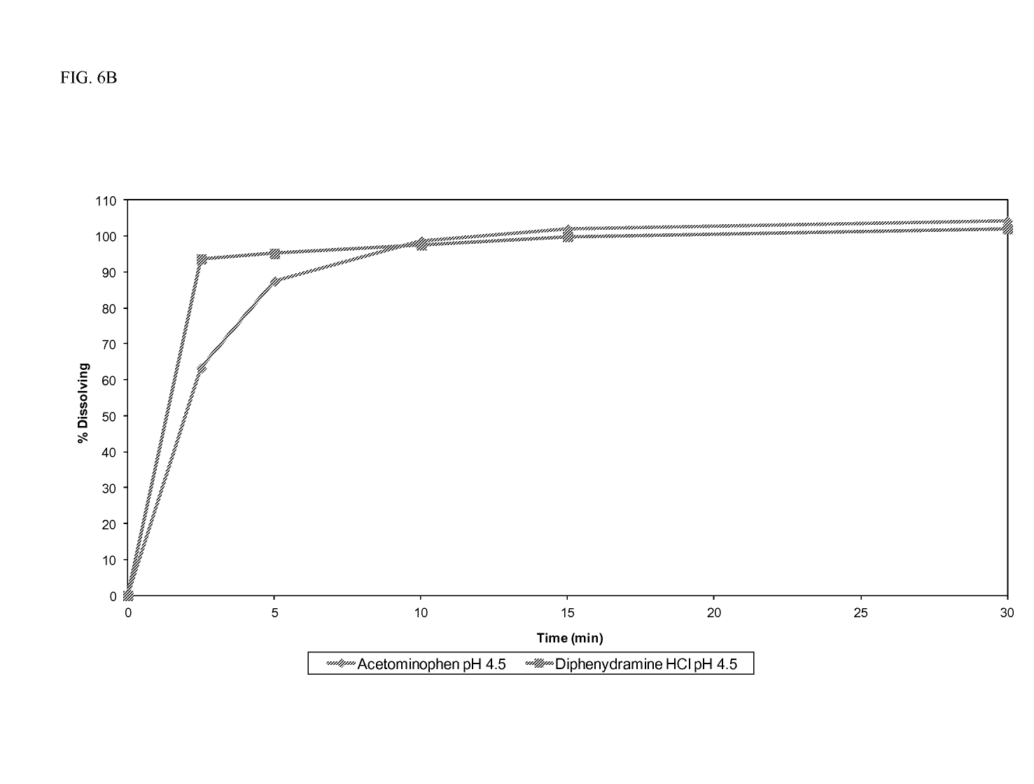

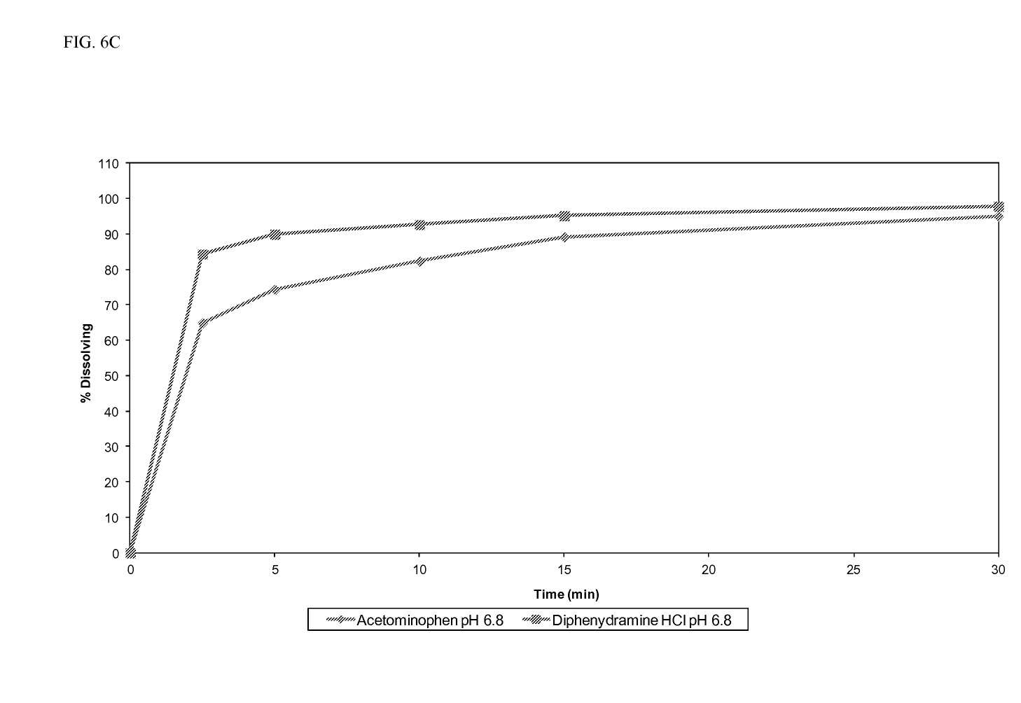

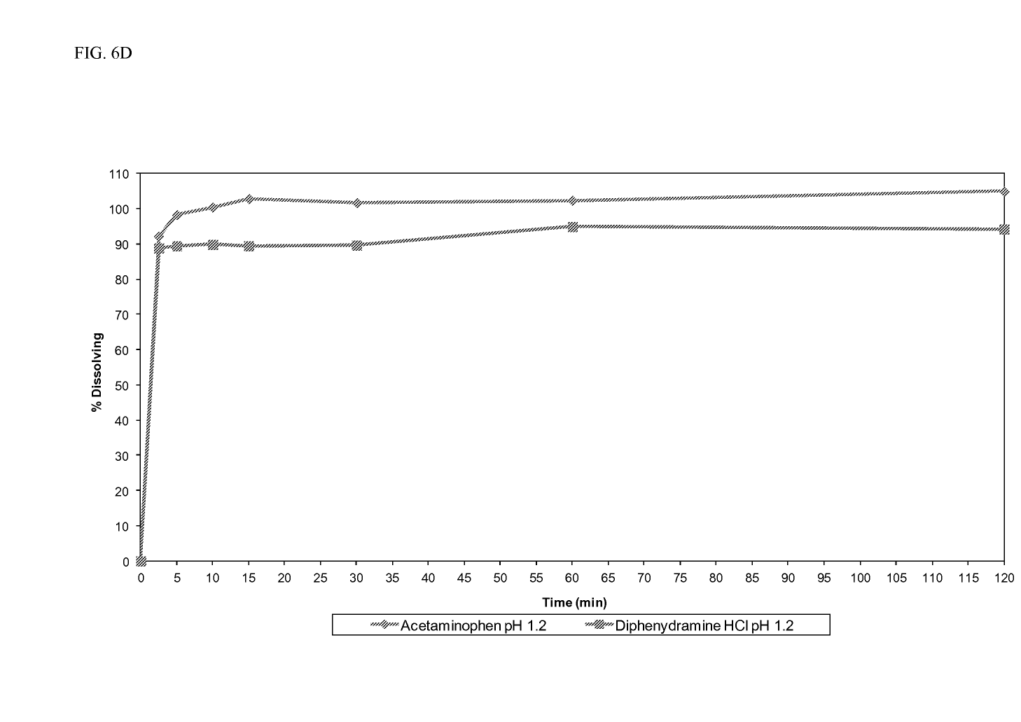

The dissolution profiles obtained on 6 units at the three pH conditions, of batch bkT027/108 and on 6 unites at pH 1.2 of batch bkT027/129 are reported in FIGS. 6A-D.

Throughout this application, various publications are referenced. The disclosures of these publications are hereby incorporated by reference in order to more fully describe the state of the art to which this invention pertains. It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1) A method of making an orally dispersible formulation comprising:
i) adding an active ingredient to a high speed mixer and shearing said active ingredient at a first rate;
ii) adding a granulating solution comprising a solvent and a coating agent to said high speed mixer while shearing said active ingredient to form a wet mixture;
iii) shearing said wet mixture in said high speed mixer at a second rate greater than said first rate;
iv) adding a diluent and a disintegrating agent and optionally other excipients (e.g. buffering agents, flavors, flavor enhancers) to said wet mixture while shearing said mixture at said second rate until a first granular mixture forms; and
v) drying said first granular mixture.

2) A method of making an orally dispersible formulation comprising:
i) adding a diluent and a disintegrating agent to a high speed mixer and shearing said disintegrating agent at a first rate;
ii) adding an active ingredient, and optionally other excipients (i.e. buffering agents, flavors, flavor enhancers) and a granulating solution while shearing said disintegrating agent at said first rate to form a wet mixture, wherein the granulating solution comprises a coating agent dissolved in a solvent;
iii) shearing said wet mixture at a second rate greater than said first rate until a first granular mixture forms; and
iv) drying said first granular mixture.

3) The method of claim 1 wherein the solvent is ethanol, water, acetone, or a combination thereof.

4) The method of claim 1 wherein said granulating solution further comprises a flavoring agent and a sweetening agent.

5) The method of claim 1 further comprising admixing said first granular mixture with a powder or second granular mixture comprising one or more flavoring agents or one or more sweetening agents or a combination thereof.

6) The method of claim 1, further comprising admixing said first granular mixture with a second granular mixture comprising at least one active ingredient that is different from that of the first granular mixture.

7) The method of claim 1, further comprising compressing said first granular mixture into tablets.

8) The method of claim 1, wherein said active ingredient is selected from the group consisting of guaifenesin, diphenhydramine, dextromethorphan, ibuprofen, ketoprofen, loratadine, phenylephrine, caffeine and acetylsalicylic acid, and combinations thereof, wherein any of said active ingredients can be present as a pharmaceutically acceptable salt.

9) The method of claim 1, further comprising the addition of said excipient, wherein the excipient is selected from the group consisting of a buffering agent, a flavoring agent, a flavor enhancer, a plasticizer, and combinations thereof.

10) An orally dispersible granular mixture comprising a plurality of granules wherein each of said granules comprises an active ingredient, a disintegrating agent, a coating agent, and optionally other excipients (e.g. buffering agents, flavors, flavor enhancers), each uniformly distributed throughout said granules, wherein said granular mixture optionally substantially completely disperses within 5 minutes in the mouth when orally administered.

11) The granular mixture of claim 10, further comprising said other excipients.

12) The granular mixture of claim 11, wherein the excipients are selected from the group consisting of a buffering agent, a flavoring agent, a flavor enhancer, a plasticizer, and combinations thereof.

13) The granular mixture of claim 9, wherein said active ingredient is selected from the group consisting of guaifenesin, diphenhydramine, dextromethorphan, ibuprofen, ketoprofen, loratadine, phenylephrine, caffeine and acetylsalicylic acid, and combinations thereof, wherein any of said active ingredients can be present as a pharmaceutically acceptable salt.

14) A method of delivering a drug to a human patient comprising administering to said patient the granular mixture of claim 9.

15) The granular mixture of claim 9, wherein said active ingredient is selected from the group consisting of guaifenesin, diphenhydramine, dextromethorphan, ibuprofen, loratadine, phenylephrine, caffeine and acetylsalicylic acid, and combinations thereof, wherein any of said active ingredients can be present as a pharmaceutically acceptable salt.
